(19) United States
(12) Patent Application Publication
(10) Pub. No.: US 2005/0181052 A1
(43) Pub. Date: Aug. 18, 2005

(54) Lansoprazole microtablets

(76) Inventors: Satishkumar Ambalal Patel, West Windsor, NJ (US); Indranil Nandi, Plainsboro, NJ (US); Suresh Palaniswamy, East Windsor, NJ (US); Pablo Davila, East Windsor, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/779,933

(22) Filed: Feb. 17, 2004

(51) Int. Cl. A61K 31/4439; A61K 9/48; A61K 9/20; A61K 9/24

(52) U.S. Cl. 424/472; 514/338

(57) ABSTRACT

A pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating.

### FIELD OF THE INVENTION

The present invention provides a pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating.

### BACKGROUND OF THE INVENTION

Proton pump inhibitors, e.g., omeprazole, lansoprazole and pantoprazole, are susceptible to degradation and/or transformation in acid medium, and thus, create a problem for formulators when it is required to provide an oral dosage form. The oral dosage form must be protected from contact with the acidic reacting gastric juice and the proton pump inhibitor must be transferred in tact form to that part of the gastrointestinal tract where pH is less acidic, neutral or alkaline and where rapid absorption of the proton pump inhibitor can occur.

Several prior art documents describe pharmaceutical compositions that are suitable for oral administration of proton pump inhibitors.

EP 0244380 describes pharmaceutical formulations containing: (a) a core in the form of small particles, i.e., pellets or compressed powder, containing the active substance along with an alkaline reacting compound; (b) one or several inert intermediate layers containing excipients for tablets which are soluble and which rapidly disintegrate in water, water-soluble film-forming polymer compounds optionally containing alkaline compounds acting as a pH buffer between the core having an alkaline reaction and the outer layer; and (c) an outer layer consisting of an enteric composition. It is also stated that, in order to improve storage stability, the cores containing the active substance should also contain constituents having an alkaline reaction, and that the water that enters by diffusion, or the gastric juice, will dissolve part of the core close to the enteric coating, forming an alkaline solution at this level inside the coated form for administration.

U.S. Pat. No. 4,786,505 describes oral dosage forms of omeprazole containing: (a) a core comprising omeprazole and an alkaline reacting compound, an alkaline salt of omeprazole and an alkaline-reacting compound or an alkaline salt of omeprazole alone; (b) at least one inert intermediate layer that is water-soluble or rapidly disintegrates in water; and (c) an external layer comprising an enteric coating.

EP A 0519144 describes a process for preparing pellets containing omeprazole in which a core constituted of inert substances is covered by the active substance in finely divided form and dispersed in an aqueous dispersion buffered to a pH of 7.0, after which an enteric coating is applied, the finished product being placed inside a capsule.

U.S. Pat. No. 5,232,706 describes pharmaceutical compositions containing: (a) a core containing omeprazole and an alkaline salt of omeprazole mixed with a first alkaline-reacting compound; (b) at least one intermediate layer formed by an excipient and a second alkaline-reacting compound; and (c) an outer layer formed by an enteric coating. It is stated that the problem of the poor stability of the omeprazole is resolved, firstly, by increasing the way the core behaves as a base either by introducing omeprazole in the form of an alkali metal or alkaline-earth salt, or a mixture of omeprazole with a basic compound or by a combination of these two possibilities; and secondly “by incorporating an intermediate layer between the core and the enteric coating for preventing the alkaline core from causing breakdown of the enteric coating”.

WO 96/01624 describes tablets containing a benzimidazole ingredient and having an enteric coating. The tablets are mixed with tablet excipients, e.g., microcrystalline cellulose, and compressed together. The resulting tablets are said to withstand acidic environment.

U.S. Pat. No. 6,248,355 describes a composition containing omeprazole which is exempt of alkaline-reacting compounds. The composition contains a core containing an acid-labile omeprazole and inert ingredients, an intermediate layer, and an enteric layer.

### SUMMARY OF THE INVENTION

The invention provides a pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8: 1, wherein said microtablets have a tablet size of about 1 mm to about 4 mm, and a tablet weight of 1 to 50 mg, and said microtablets are free of a separating or intermediate layer between the lansoprazole and enteric coating.

According to another aspect, the invention provides a method for treating gastrointestinal sicknesses comprising administering to a patient in need thereof a pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating.

### DETAILED DESCRIPTION OF THE INVENTION

The invention provides a pharmaceutical composition comprising microtablets, wherein said microtablets comprise lansoprazole, a lubricant, optionally one or more excipients, and an enteric coating. As used herein, “lansoprazole” is 2-[[[3-methyl-4-(2,2,2-trifluoroethoxy)-2-pyridyl] methyl] sulfinyl] benzimidazole and includes salts (hydrates, etc.), esters and the like (including pro-drugs). The microtablets are free of a separating or intermediate layer between the lansoprazole and enteric coating. The weight ratio of lansoprazole to lubricant is from about 1:4 to about 8:1, preferably, from about 1:2 to about 5:1, more preferably about 1:1.

The microtablets are cylindrical with a flat or convex upper side and lower side and with a diameter and height which are preferably approximately equal and, independently of one another, preferably have a tablet size of from about 1 mm to about 4 mm, more preferably from about 1.5 mm to about 2.5 mm. The microtablets have a tablet weight of about 1 mg to about 50 mg. Preferably, the microtablets have a tablet weight of about 2 mg to about 10 mg.

The lubricant is preferably selected from calcium stearate, magnesium stearate, sodium stearate, zinc stearate, glyceryl monostearate, glyceryl palmitostearate, hydrogenated castor oil, hydrogenated vegetable oil, light mineral oil, mineral oil, polyethylene glycol, sodium benzoate, sodium lauryl sulfate, sodium stearyl fumarate, stearic acid and talc. A combination of lubricants may also be used. A preferred lubricant is magnesium stearate.

The microtablets of the invention are preferably free of an alkaline-reacting compound. As used herein, “free of an alkaline-reacting compound” means that the microtablets are essentially or substantially free of any alkaline-reacting compound. In other words, microtablets in which the amount of alkaline-reacting compound is not sufficient to set up an alkaline micro-environment around the lansoprazole when it is in contact with an acid or neutral aqueous medium, e.g., a micro-environment having a pH above 7.

The usual oral recommended dose of lansoprazole for humans is between about 15 mg/day and about 60 mg/day and this dose may be administered in two or three divided doses, preferably with food if administered orally. A maximum recommended daily dose for humans would be about 350 mg, but it will be understood by one skilled in the art that dosage under this invention will be determined by the particular circumstances surrounding each case.

The lansoprazole is present in an amount of from about 2 to about 50 weight percent (%), based on the total weight of the microtablet. Preferably, the lansoprazole is present in an amount of from about 5 to about 20 weight percent, based on the total weight of the microtablet.

The enteric coating is preferably prepared using a water-insoluble polymer. The water-insoluble polymer may display pH-independent solubility and may comprise a water-insoluble polymer mixture. As used herein, “water-insoluble”, means a polymer solubility in water at room temperature of less than 100 mg/L, e.g. 20 mg/L or less, e.g., 10 mg/L or less, e.g., 1 mg/L or less. The enteric coating is applied to the microtablets by conventional coating techniques, such as coating in a tank or a fluidized bed employing polymer solutions in water or in suitable organic solvents or using latex suspensions of these polymers.

Preferred enteric coatings include the following: cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene copolymer; polyvinylalcohols; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g., EUDRAGIT®-L and -S series, such as L100-55, L30D55, L100, S100, L12.5 and S12.5, available from Rohm; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane. A combination of enteric coatings may also be used.

More preferably, the enteric coating is selected from a copolymer of methacrylic acid and methyl methacrylate, and a copolymer of methacrylic acid and ethyl acrylate. Most preferably, the enteric coating is poly(methacrylic acid, ethyl acrylate)1:1 (EUDRAGIT®-L30D 55 and EUDRAGIT®-L100-55).

The enteric coating is present in an amount of from about 5 to about 50 weight percent (%), based on the total weight of the microtablet. Preferably, the enteric coating is present in an amount of from about 15 to about 20 weight percent, based on the total weight of the microtablet.

It is within the scope of the invention for the microtablets to include one or more pharmaceutically acceptable excipients. Examples of such excipients are binders, diluents, plasticizers, anti-caking agents, fillers, solubilizing agents, disintegrants, surfactants, flavorants, sweeteners, stabilizers, anti-oxidants, anti-adherents, preservatives, glidants and pigments. A combination of excipients may also be used. Such excipients are known to those skilled in the art, and thus, only a limited number will be specifically referenced.

Preferred binders include, but are not limited to, starches, e.g., potato starch, wheat starch and corn starch; gums, such as gum tragacanth, acacia gum and gelatin; and polyvinyl pyrrolidone, e.g., Povidone. Polyvinyl pyrrolidone is a particularly preferred binder.

Preferred plasticizers include, but are not limited to, citric and tartaric acid esters, (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates, such as Polysorbar 50); sorbitan monooleate. A combination of plasticizers may also be used. A preferred plasticizer is triethyl citrate or a combination of triethyl citrate and glycerol monostearate.

The enteric coating component of the invention preferably contains a plasticizer. The amount of plasticizer is in general optimized for each enteric coating polymer and generally represents from about 1 weight percent (%) to about 50 weight percent, preferably 2 to 20 weight percent, based on the total weight of the enteric coating polymer.

Preferred fillers include, but are not limited to, microcrystalline cellulose, starch, pregelatinized starch, modified starch, dibasic calcium phosphate dihydrate, calcium sulfate trihydrate, calcium sulfate dihydrate, calcium carbonate, dextrose, sucrose, lactose, mannitol and sorbitol. Lactose is a particularly preferred filler.

Examples of disintegrants include:
(i) natural starches, such as maize starch, potato starch and the like, directly compressible starches, e.g., Sta-rx® 1500; modified starches, e.g., carboxymethyl starches and sodium starch glycolate, available as Primojel®, Explotab®, Explosol®; and starch derivatives, such as amylose;
(ii) cross-linked polyvinylpyrrolidones, e.g., crospovidones, such as Polyplasdone® XL and Kollidon® CL;
(iii) alginic acid and sodium alginate;
(iv) methacrylic acid-divinylbenzene co-polymer salts, e.g., Amberlite® IRP-88; and
(v) cross-linked sodium carboxymethylcellulose, available as, e.g., Ac-di-sol®, Primellose®, Pharmacel® XL, Explocel® and Nymcel® ZSX.
   Additional disintegrants also include hydroxypropyl cellulose, hydroxypropylmethyl cellulose, croscarmellose sodium, sodium starch glycolate, polacrillin potassium, polyacrylates, such as Carbopol®, magnesium aluminium silicate and bentonite.

Examples of surfactants include:
1) Reaction products of a natural or hydrogenated castor oil and ethylene oxide.

The polyethyleneglycol-hydrogenated castor oils available under the trademark CREMOPHOR are especially suitable, such as CREMOPHOR RH 40 and CREMOPHOR RH 60. Also suitable are polyethyleneglycol castor oils, such as that available under the trade name CREMOPHOR EL.
2) Polyoxyethylene-sorbitan-fatty acid esters, also called polysorbates, e.g., mono-and tri-lauryl, palmityl, stearyl and oleyl esters of the type known and commercially-available under the trademark TWEEN.
   20 [polyoxyethylene(20)sorbitanmonolaurate],
   21 [polyoxyethylene(4)sorbitanmonolaurate],
   40 [polyoxyethylene(20)sorbitanmonopalmitate],
   60 [polyoxyethylene(20)sorbitanmonostearate],
   65 [polyoxyethylene(20)sorbitantristearate],
   80 [polyoxyethylene(20)sorbitanmonooleate],
   81 [polyoxyethylene(5)sorbitanmonooleate],
   85 [polyoxyethylene(20)sorbitantrioleate].
   A preferred product of this class is TWEEN 80.
   Although polyethylene glycol (PEG) itself does not function as a surfactant, a variety of PEG-fatty acid esters have useful surfactant properties. Among the PEG-fatty acid monoesters, esters of lauric acid, oleic acid and stearic acid are most useful. Among the surfactants of Table 1, preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate.
3) Polyoxyethylene fatty acid esters, for example polyoxyethylene stearic acid esters of the type known and commercially available under the trademark MYRJ.
4) Polyoxyethylene-polyoxypropylene co-polymers and block co-polymers, e.g., of the type known and commercially-available under the trademark PLURONIC, EMKALYX and POLOXAMER. Preferred products of this class are PLURONIC F68 and POLOXAMER 188.
5) Dioctylsulfosuccinate or di-[2-ethylhexyl]-succinate.
6) Phospholipids, in particular, lecithins. Suitable lecithins include, in particular, soybean lecithins.
7) Propylene glycol mono- and di-fatty acid esters, such as propylene glycol dicaprylate (also known and commercially-available under the trademark MIGLYOL 840), propylene glycol dilaurate, propylene glycol hydroxystearate, propylene glycol isostearate, propylene glycol laurate, propylene glycol ricinoleate and propylene glycol stearate.
8) Polyoxyethylene alkyl ethers, such as those commercially-available under the trademark BRIJ, e.g., Brij 92V and Brij 35.
9) Tocopherol esters, e.g., tocopheryl acetate and tocopheryl acid succinate.
10) Docusate salts, e.g., dioctylsulfosuccinate or related compounds, such as di-[2-ethylhexyl]-succinate.

A combination of surfactants may also be used.

Preferred sweeteners include, but are not limited to, artificial sweeteners, such as aspartame, saccharin and cyclamates; natural sweeteners, such as sucrose, fructose, glucose, lactose, maltodextrin and sodium glycolate; and mixtures of artificial and natural sweeteners, such as a mixture of aspartame and sucrose.

Preferred flavorants include, but are not limited to, cherry, strawberry, fruit punch, grape, cream, vanilla, chocolate, mocha, spearmint, cola and the like.

Preferred pigments include, but are not limited to, titanium dioxide, iron oxide and vegetable dyes.

Preferred diluents include, but are not limited to, dextrose, sorbitol, sucrose, lactose, mannitol, urea, potassium chloride, sodium chloride, gelatin, starch, methyl cellulose, ethyl cellulose, propyl cellulose, hydroxymethyl cellulose, hydroxyethyl cellulose, hydroxypropyl cellulose, hydroxypropyl methyl cellulose, silica, polyvinyl alcohol, polyvinylpyrrolidone and magnesium stearate.

The microtablets of the invention are preferably prepared by mixing lazoprasole and a lubricant, and optionally one or more excipients, in the presence or absence of a solvent, to form a premix. The premix is preferably in the form of a solid dispersion or a homogeneous suspension. The premix is preferably subject to granulation, melt extrusion, wet granulation or roller compaction, to form microtablets. The microtablets are preferably dried, or cooled in the case of melt extrusion, and optionally milled and/or screened. The tabletting takes place in a suitable tabletting machine equipped with multiple microtablet punches. The microtablets are coated with an enteric coating.

Useful drying techniques include spray-drying, fluid bed drying, flash drying, ring drying, micron drying, tray drying, vacuum drying, radio-frequency drying and microwave drying. A preferred drying technique is fluid bed. Useful mills include fluid energy mill, ball mill or rod mill, hammer mill, cutting mill and oscillating granulator. More specifically, suitable mills include, Quadro, Fryma, Glatt Quick Sieve, Fluidaire, Fitzpatrick (Fitz mill), BTS mill and Tornado. A preferred mill is a Fitz mill.

In one embodiment of the invention, the microtablets are enclosed inside a capsule, for example, a gelatin capsule. For this, any gelatin capsule conventionally employed in the pharmaceutical formulation field can be used, such as the hard gelatin capsule known as CAPSUGEL®.

The microtablets of the invention are particularly suitable for oral administration of lanzoprasol and are particularly suitable for treating gastrointestinal sicknesses.

The following non-limiting examples illustrate further aspects of the invention.

### EXAMPLE 1

| | | Quantity per | |
|---|---|---|---|
| Ingredient | % | Capsule* (mg) | |
| Premix: | | | |
| Lansoprazole | 12.75 | 30 | |
| Magnesium Stearate, NF | 9.35 | 22 | |
| Lactose, regular, NF | 51 | 120 | |
| Sodium Starch Glycolate, NF | 7.65 | 18 | |
| HPMC E 5LV, USP | 3.4 | 8 | |
| Final Mix: | | | |
| Magnesium Stearate, NF | 0.85 | 2 | |
| TOTAL | | 200 | mg |
| Tablet Coating: | | | |
| Eudragit L30D 55, NF | 8.5 | 20 | |
| Methacrylic ester copolymer | | | |
| Talc, Low Micron, USP | 2.26 | 5.33 | |
| Triethyl Citrate, NF | 1.27 | 3 | |
| Magnesium Stearate, NF | 2.13 | 5 | |
| Simethicone, USP | 0.76 | 1.8 | |
| Sodium Lauryl Sulfate, NF | 0.09 | 0.2 | |
| Purified water, USP | none | q.s. | |
| TOTAL | 100% | 235.32 | mg |

*Each capsule contains approximately 40 microtablets on a theoretical weight basis.

A pre-mix was prepared by mixing lansoprazole, magnesium stearate, lactose, sodium starch glycolate, and hydroxypropyl methyl cellulose, in a high shear mixer to form a wet granulation. The wet granulation was dried using a GPGC Fluid Bed Dryer. The granules were milled using a Fitz-mill equipped with 0.033 inch screen. Magnesium stearate was added and blended with the dried granules using a Patterson Kelly Twin Shell Blender. The blend was compressed using a Rotary Tablet Press equipped with 0.0787 inch diameter deep concave tooling and compression force measurement. Tooling is nine 2 mm diameter carbide tips per punch in a circular configuration to yield microtablets having a diameter of 2 mm. The microtablets were produced in a rotary tabletting machine equipped with multiple microtablet punches.

The coating which was prepared by dissolving or dispersing Eudragit L30 D55 in water, adding talc, triethyl citrate, magnesium stearate, simethicone, and sodium lauryl sulfate, with mixing. The coating was deposited onto the microtablets using a Vector LDCS partially perforated coating pan.

### EXAMPLE 2

Microtablets prepared in Example 1 were encapsulated into three hard gelatin capsules. separately, microtablets prepared in Example 1 were dried in the Vector LDCS partially perforated coating pan and then encapsulated into three hard gelatin capsules. The capsules were filled to contain approximately 40 microtablets on a theoretical weight basis. Separately, six capsules of PREVACID, available from TAP Pharmaceuticals Inc., were evaluated each of which contained 30 mg of lasoprazole, hydroxypropyl cellulose, low substituted hydroxypropyl cellulose, colloidal silicon dioxide, magnesium carbonate, methacrylic acid copolymer, starch, talc, sugar sphere, sucrose, polyethylene glycol, polysorbate 80, and titanium dioxide. PREVACID capsules contain granules having a barrier coat and an enteric coating.

Each of the above-described capsules was evaluated in a Phase I and a Phase II dissolution study. In Phase I, each capsule was placed in 500 mL 0.1 N HCL using USP Apparatus 2 at a paddle speed of 75 rmp for 60 minutes. In Phase II, each capsule was placed in a mixture containing 425 mL of phosphate buffer and 475 mL of the solution from Phase I for a total of 900 mL at a pH of 6.8 in a USP Apparatus 2 at a paddle speed of 75 rpm for 90 minutes. Samples were taken according to the schedule set forth in Table I and the amount of lansoprazole was determined by UV. An average dissolution profiles for the three capsules containing dried and undried microtablets according to the invention, and six capsules containing PREVACID, are summarized in Table 1.

TABLE 1

| | PREVACID | Dried | Undried |
|---|---|---|---|
| | Capsules | Microtablets | Microtablets |
| | Avg. Drug | Avg. Drug | Avg. Drug |
| Time (min) | Dissolved | Disolved | Dissolved |
| Phase I | | | |
| 0 | 0 | 0 | 0 |
| 60 | 0.6 | 0.2 | 0.2 |
| Phase II | | | |
| 5 | 70.5 | 100.3 | 81.5 |
| 10 | 92.4 | 98.7 | 97.1 |
| 15 | 95.8 | 99.7 | 100.7 |
| 30 | 100.7 | 101.1 | 98.8 |
| 45 | 102.3 | 100.5 | 99.6 |
| 60 | 103.1 | 99.6 | 99.3 |
| 90 | 105.4 | 99.6 | 99.5 |

The results in Table I clearly show that capsules containing the microtablets prepared according to the invention display uniform dissolution which is equivalent to the commercially available PREVACID.

While the invention has been described with particular reference to certain embodiments thereof, it will be understood that changes and modifications may be made by those of ordinary skill within the scope and spirit of the following claims:

1. A pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating.

2. A pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating, and said microtablets are free of an alkaline-reacting compound.

3. The composition according to claim 1, wherein the weight ratio of lansoprazole to lubricant is from about 1:2 to about 5:1.

4. The composition according to claim 3, wherein the weight ratio of lansoprazole to lubricant is about 1:1.

5. The composition according to claim 1, wherein the lansoprazole is present in an amount of from about 2 to about 50 weight percent, based on the total weight of the microtablet.

6. The composition according to claim 5, wherein the lansoprazole is present in an amount of from about 5 to about 20 weight percent, based on the total weight of the microtablet.

7. The composition according to claim 1, wherein the lubricant is selected from the group consisting of calcium stearate, magnesium stearate, sodium stearate, zinc stearate, glyceryl monostearate, glyceryl palmitostearate, hydrogenated castor oil, hydrogenated vegetable oil, light mineral oil, mineral oil, polyethylene glycol, sodium benzoate, sodium lauryl sulfate, sodium stearyl fumarate, stearic acid, talc and combinations thereof.

8. The composition according to claim 7, wherein the lubricant is magnesium stearate.

9. The composition according to claim 1, wherein the microtablets have a tablet size of about 1.5 mm to about 2.5 mm.

10. The composition according to claim 1, wherein the microtablets have a tablet weight of 2 to 10mg.

11. The composition according to claim 1, wherein the enteric coating is selected from the group consisting of cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene co-polymer; polyvinylalcohols; copolymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, and octadecyl acrylate; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid copolymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane and combinations thereof.

12. The composition according to claim 11, wherein the enteric coating is selected from the group consisting of a co-polymer of methacrylic acid and methyl methacrylate, and a copolymer of methacrylic acid and ethyl acrylate.

13. The composition according to claim 12, wherein the enteric coating is poly(methacrylic acid, ethyl acrylate).

14. The composition according to claim 1, wherein the enteric coating is present in an amount of from about 5 to about 50 weight percent, based on the total weight of the microtablet.

15. The composition according to claim 1, wherein the enteric coating is present in an amount of from about 15 to about 20 weight percent, based on the total weight of the microtablet.

16. The composition according to claim 1, wherein the enteric coating additionally comprises at least one plasticizer.

17. The composition according to claim 16, wherein the plasticizer is selected from the group consisting of acetyl-triethyl citrate, acetyl tributyl-, tributyl-, triethyl-citrate, glycerol diacetate, glycerol triacetate, acetylated monoglycerides, castor oil, dibutyl-phthalate, diamyl-phthalate, diethyl-phthalate, dimethyl-phthalate, dipropyl-phthalate, di-(2-methoxy- or 2-ethoxyethyl)-phthalate, ethylphthalyl glycolate, butylphthalylethyl glycolate, butylglycolate, propylene glycol, polyethylene glycol, diethyladipate, di-(2-methoxy- or 2-ethoxyethyl)-adipate, benzophenone, diethyl- and diburylsebacate, dibutylsuccinate, dibutyltartrate, diethylene glycol dipropionate, ethyleneglycol diacetate, ethyleneglycol dibutyrate, ethyleneglycol dipropionate, tributyl phosphate, tributyrin, polyethylene glycol sorbitan monooleate, sorbitan monooleate, and combinations thereof.

18. The composition according to claim 17, wherein the plasticizer is triethyl citrate and glycerol monostearate.

19. The composition according to claim 1, which additionally comprises at least one excipient.

20. The composition according to claim 19, wherein the excipient is independently selected from the group consisting of a binder, diluent, plasticizer, anti-caking agent, filler, solubilizing agent, disintegrant, surfactant, flavorant, sweetener, stabilizer, anti-oxidant, anti-adherent, preservative, glidant, pigment and combinations thereof.

21. A method for treating gastrointestinal sicknesses comprising administering to a patient in need thereof a pharmaceutical composition comprising microtablets, wherein said microtablets comprise lansoprazole, a lubricant, optionally one or more excipients, and an enteric coating, wherein the weight ratio of lansoprazole to lubricant is from about 1:4 to about 8:1, wherein said microtablets have a tablet size of about 1 mm to about 4 mm, and a tablet weight of 1 to 50 mg, and said microtablets are free of a separating or intermediate layer between the lansoprazole and enteric coating.
